(19) United States
(12) Patent Application Publication
(10) Pub. No.: US 2025/0138026 A1
(43) Pub. Date: May 1, 2025

(54) METHOD FOR DIAGNOSING COLLAGEN DEGRADATION ASSOCIATED DISEASE

(71) Applicants: INSTITUT NATIONAL DE LA SANTÉ ET DE LA RECHERCHE MÉDICALE, Paris (FR); HOSPICES CIVILS DE LYON, Lyon (FR); UNIVERSITÉ CLAUDE BERNARD LYON 1, Villeurbanne (FR)

(72) Inventors: Roland CHAPURLAT, Lyon (FR); Patrick GARNERO, Lyon (FR); Evelyne GINEYTS, Lyon (FR)

(21) Appl. No.: 18/834,001

(22) PCT Filed: Jan. 31, 2023
(86) PCT No.: PCT/EP2023/052323

§ 371 Date: Jul. 29, 2024

(30) Foreign Application Priority Data
Feb. 1, 2022 (EP) 22305111.1

(51) Int. Cl. G01N 33/68; A61K 45/06

(52) U.S. Cl. CPC G01N 33/6887; A61K 45/06; G01N 2333/78; G01N 2470/10; G01N 2800/102; G01N 2800/105; G01N 2800/52

(57) ABSTRACT

The Inventors have developed an ELISA of a new molecular marker detecting a neoepitope generated from the cleavage of the α1 chain of type III collagen within its helical domain. Serum levels of this marker were significantly increased in patients with RA and is significantly associated to CRP and ESR levels. Indeed, they demonstrated that the median serum HELIX-III levels were significantly higher in patients with moderate (p=0027) and active RA (p=00004) compared with those in age-matched controls. The present invention relates to an antibody recognizing an epitope having SEQ ID NO:1 of collagen protein and its uses for diagnostic, prognostic and monitoring purposes.

### FIELD OF THE INVENTION

The present invention provides methods and means for diagnosing and predicting collagen degradation associated disease and treatment response.

### BACKGROUND OF THE INVENTION

Collagen degradation corresponds to a metabolic breakdown of collagen. Small breakdown fragments are measured in the circulation or in the urine. While collagen degradation is a normal part of collagen homeostasis, excessive collagenolysis has been implicated in a number of human diseases such as arthritis, cancer, atherosclerosis and hepatic fibrosis.

One of the major clinical manifestations of rheumatoid arthritis (RA) is severe joint damage resulting in disability (1). The mechanisms leading to joint damage are not yet fully understood, but synovial tissue is activated with the secretion of pro-inflammatory cytokines such as interleukin 1 and tumor necrosis factor α, and enzymes such as MMPs or other catabolic factors ultimately inducing cartilage destruction and bone erosion (2, 3). Monitoring of RA patients consists in assessing disease activity, using quantification of pain, swollen joints and inflammation is done by noting pain and reduced mobility resulting from joint destruction. However, these metrics are not specific for joint diseases and poorly correlated with joint destruction (5, 6). The availability of specific markers reflecting the renewal of the extracellular matrix of the synovium, cartilage and bone is essential for a better understanding of the pathophysiology of articular tissues. For about twenty years, specific biological markers of bone tissue have shown their-utility for the evaluation of remodeling in various bone pathologies. such as, for example, PINP and osteocalcin for bone formation and CTX-I or NTX-I for bone resorption. Biochemical markers to monitor cartilage turnover have been developed such as collagen terminal crosslinking type II telopeptide (CTX-II) (7, 8). Increased levels of enzyme-linked immunosorbent (ELISA)-assayed CTX-II have been reported in the urine of patients with RA and the elevated levels are associated with more rapid progression of joint destruction in RA (9, 10).

In RA, the normal thin synovial membrane proliferates due to the activation of the surrounding synoviocytes and fibroblasts which will synthesize an abnormally thick collagen matrix with numerous villi, with an extensive vascularization and an infiltration of numerous inflammatory cells (monocytes/macrophages, dendritic cells, lymphocytes and polymorphonuclear neutrophils) and which will eventually form a pannus (2, 11). The bordering cells produce many catalytic factors (MMP, cytokines) which will contribute to the destruction of the cartilage and then to the exposure and destruction of the subchondral bone. The extracellular matrix of the synovial is composed of collagens, fibronectin, vitronectin, laminin, tenascin, proteoglycans and elastin (12). The collagens are essentially of fibrillar type I, II and type III and also type IV, V and VI (13-16). High baseline levels of Glucosyl Galactosyl Pyridinoline (Glu-Gal-PYD), which is a crosslinking of synovium fibrillar collagens has been shown to be associated with a risk increased of progression of joint destruction in early RA (17). However, Glu-Gal-PYD is measured by HPLC technique which it does not currently make it a widely used marker for assessing synovial tissue damage (18).

### SUMMARY OF THE INVENTION

The invention relates to an antibody recognizing an epitope having SEQ ID NO:1 of collagen protein and its uses for diagnostic and monitoring purposes.

In particular, the invention is defined by claims.

### DETAILED DESCRIPTION OF THE INVENTION

Inventors have developed an ELISA of a new molecular marker detecting a neoepitope generated from the cleavage of the α1 chain of type III collagen within its helical domain. Serum levels of this marker, measured by ELISA were significantly increased in patients with RA and were significantly associated to C Reactive Protein (CRP) and erythrocyte sedimentation rate (ESR) levels (r=0.59, P<0.0001 and r=0.44, P=0.001 respectively). Indeed, they demonstrated that the median serum Helix-III levels were significantly higher in patients with moderate (p=0027) and active RA (p=00004) compared with those in age-matched controls.

Accordingly, the serum assay Helix-III of degradation marker of type III collagen can provide information on synovial damage.

In a first aspect, the invention relates to an antibody recognizing an epitope having SEQ ID NO:1 of collagen protein. In some embodiments, the invention relates to an antibody recognizing an epitope consisting to SEQ ID NO:1 of collagen protein.

As used herein, the term “antibody” has its general meaning in the art and refers to any antibody-like molecule that has an antigen binding region, and this term includes antibody fragments that comprise an antigen binding domain such as Fab′, Fab, F(ab′)2, and single domain antibodies (DABs). In natural antibodies, two heavy chains are linked to each other by disulfide bonds and each heavy chain is linked to a light chain by a disulfide bond. There are two types of light chain, lambda (1) and kappa (κ). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hypervariable or complementarity determining regions (CDRs). Occasionally, residues from nonhypervariable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs.

The term “antibody fragment” refers to at least one portion of an intact antibody, preferably the antigen binding region or variable region of the intact antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, single chain antibody molecules, in particular scFv antibody fragments, disulfide-linked Fvs (sdFv), a Fd fragment consisting of the VH and CHI domains, linear antibodies, single domain antibodies such as, for example, sdAb (either VL or VH), camelid VHH domains, multi-specific antibodies formed from antibody fragments such as, for example, a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type III (see U.S. Pat. No. 6,703,199, which describes fibronectin polypeptide minibodies). Papain digestion of antibodies produces two identical antigen-binding fragments, called “Fab” fragments, and a residual “Fc” fragment, a designation reflecting the ability to crystallize readily.

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH:VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

In a particular embodiment, the antibody according to the invention which is a polyclonal antibody or a monoclonal antibody.

In a particular embodiment, the antibody according to the invention which is a Fab′, Fab, F(ab′)2, scFv or a single domain antibody.

According to the present invention, the antibody of the present invention has specificity for an epitope of collagen protein. In some embodiments, the epitope of collagen protein is derived from a collagen protein.

In a particular embodiment, the epitope of collagen protein is called Helix-III.

As used herein, the term “specificity” refers to the ability of an antibody to detectably bind to an epitope presented on collagen protein. More particularly, the antibody according to the invention has specificity for Helix-III protein or peptide.

As used herein, the term “epitope” also called as “Helix-IIT peptide” in the context of the invention refers to a specific arrangement of amino acids located on a protein to which an antibody binds. Epitopes often consist of a chemically active surface grouping of molecules such as amino acids or sugar side chains, and have specific three dimensional structural characteristics as well as specific charge characteristics. Epitopes can be linear or conformational, i.e., involving two or more sequences of amino acids in various regions of the antigen that may not necessarily be contiguous.

As used herein, the term “recognizing” or “binding” refers to a direct association between two molecules, due to, for example, covalent, electrostatic, hydrophobic, and ionic and/or hydrogen-bond interactions, including interactions such as salt bridges and water bridges. In particular, as used herein, the term “binding” in the context of the binding of an antibody to a predetermined antigen or epitope typically is a binding with an affinity corresponding to a K_{D} of about 10⁻⁷ M or less, such as about 10⁻⁸ M or less, such as about 10⁹ M or less, about 10⁻¹⁰ M or less, or about 10⁻¹¹ M or even less.

The terms “peptide”, “polypeptide”, and “protein” are used interchangeably, and refer to a compound comprised of amino acid residues covalently linked by peptide bonds. A polypeptide is not limited to a specific length: it must contain at least two amino acids, and no limitation is placed on the maximum number of amino acids that can comprise a polypeptide's sequence. Peptides, oligopeptides, and proteins are included within the definition of polypeptide, and such terms may be used interchangeably herein unless specifically indicated otherwise. As used herein, the term refers to both short chains, which also commonly are referred to in the art as peptides, oligopeptides and oligomers, for example, and to longer chains, which generally are referred to in the art as proteins, of which there are many types. In one embodiment, as used herein, the term “peptides” refers to a linear polymer of amino acids linked together by peptide bonds, preferably having a chain length of less than about 50 amino acids residues; a “polypeptide” refers to a linear polymer of at least 50 amino acids linked together by peptide bonds; and a protein specifically refers to a functional entity formed of one or more peptides or polypeptides, optionally glycosylated, and optionally of non-polypeptides cofactors. This term also does exclude post-expression modifications of the polypeptide, for example, glycosylations, acetylations, phosphorylations and the like, as well as other modifications known in the art, both naturally occurring and non-naturally occurring. A polypeptide may be an entire protein, or a subsequence thereof. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

As used herein, the term “Helix-III peptide” refers to a neoepitope generated from the cleavage of the collagen.

As used herein, the term “collagen” refers to the main structural protein in the extracellular matrix found in the body's various connective tissues. The collagen protein is composed of a triple helix, which generally consists of two or three identical chains (α1) and possibly an additional chain that differs slightly in its chemical composition (α2). The amino acid composition of collagen is atypical for proteins, particularly with respect to its high hydroxyproline content. The most common motifs in the amino acid sequence of collagen are glycine-proline-X and glycine-X-hydroxyproline, where X is any amino acid other than glycine, proline or hydroxyproline.

In a particular embodiment, Helix-III peptide is an epitope generated from the cleavage of α1 chain of type III collagen within its helical domain. In some embodiments, the epitope of collagen protein is derived from the sequence of the α1 chain of human type III collagen.

Type III Collagen is a homotrimer, or a protein composed of three identical peptide chains (monomers), each called an alpha 1 chain of type III collagen. Formally, the monomers are called collagen type III, alpha-1 chain (COL3A1) and in humans are encoded by the COL3A1 gene. The naturally occurring human COL3A1) gene has a nucleotide sequence as shown in Genbank Accession number NM_000090, NM_001376916 and the naturally occurring human COL3A1 protein has an amino acid sequence as shown in Genbank Accession number NP_000081. The murine nucleotide and amino acid sequences have also been described (Genbank Accession numbers NM_009930 and NP_034060).

In a particular embodiment, Helix-III peptide is a specific fragment (epitope) resulting from the degradation of the helix domain of type III collagen, type IV and XI In a particular embodiment, the Helix-III peptide has at least the following amino acid sequence: SEQ ID NO: 1.

In a particular embodiment, the Helix-III peptide has the following amino acid sequence: SEQ ID NO: 2.

In a particular embodiment, the Helix-III peptide has the following amino acid sequence: SEQ ID NO: 3.

In a particular embodiment, the Helix-III peptide has the following amino acid sequence: SEQ ID NO: 4.

In a particular embodiment, the Helix-III peptide has the following amino acid sequence: SEQ ID NO: 5.

In a particular embodiment, the Helix-III peptide has the following amino acid sequence: SEQ ID NO: 6.

In a particular embodiment, the Helix-III peptide has the following amino acid sequence: SEQ ID NO: 7.

In a particular embodiment, the Helix-III peptide has the following amino acid sequence: SEQ ID NO: 8.

In a particular embodiment, the epitope or Helix-III peptide comprises or consists of the amino acid sequences SEQ ID NO: 1, SEQ ID NO: 2, SEQ ID NO: 3, SEQ ID NO: 4, SEQ ID NO: 5, SEQ ID NO: 6, SEQ ID NO:7, SEQ ID NO: 8. In some embodiment, the epitope consists of the amino acid sequences SEQ ID NO: 1, SEQ ID NO: 2, SEQ ID NO: 3, SEQ ID NO:7 or SEQ ID NO:8. In some embodiment, the epitope consists of the amino acid sequences SEQ ID NO:2, SEQ ID NO: 3, SEQ ID NO:7 or SEQ ID NO:8. In some embodiments, the epitope consists to the amino acid sequence SEQ ID NO:2.

In a particular embodiment, the collagen degradation products hydroxyproline (OHPr), hydroxlysine glycosides, the 3-hydroxy-pyridinium crosslinks pyridinoline (PYD), and deoxypyridinoline (DPD) or their higher-molecular-weight derivates originating from the nonhelical (i.e. amino-terminal cross-linked telopeptide (NTX-I), carboxy-terminal cross-linked telopeptides (CTX-I) and helical (helical peptide; HELP)) region of the collagen type I molecule.

TABLE A
Epitope sequences detected by the antibody according to the invention

| SEQ ID NO: | Sequences |
|---|---|
| SEQ ID NO: 1 | PGPPGTS |
| SEQ ID NO: 2 | 178-PPGPPGPhypGTS-188 (where hyp is hydroxyproline) |
| SEQ ID NO: 3 | PPGPPGPPGTS (Col-III(α1) non hydroxylated peptide |
| SEQ ID NO: 4 | PPGPPGPhypGT (Col-III(α1) peptide − 1 amino acid) |
| SEQ ID NO: 5 | PPGPPGPhypGTSG (Col-III(α1) peptide + 1 amino acid) |
| SEQ ID NO: 6 | PSGPPGKDGTS (analogous Col-III peptide) |
| SEQ ID NO: 7 | LQGPPGPPGTS (Col-IV(α5) peptide |
| SEQ ID NO: 8 | SDGAPGPPGTS (Col- XI(α2) peptide) |

In some embodiments, the antibody of the invention binds to an epitope comprising 7 amino acid residues from SEQ ID NO:1.

In a particular embodiment, the antibody according to the invention binds to SEQ ID NO: 2, SEQ ID NO: 3, SEQ ID NO: 4, SEQ ID NO: 5, SEQ ID NO: 6, SEQ ID NO:7, SEQ ID NO: 8. In some embodiments, the antibody according to the invention binds to a sequence consisting to SEQ ID NO: 2, SEQ ID NO: 3, SEQ ID NO: 4, SEQ ID NO: 5, SEQ ID NO: 6, SEQ ID NO:7 or SEQ ID NO: 8. In some embodiments, the antibody according to the invention binds to a sequence consisting to SEQ ID NO: 2, SEQ ID NO: 3, SEQ ID NO: 7 or SEQ ID NO:8. In a preferred embodiment, the antibody according to the invention binds to SEQ ID NO:2. In some embodiments, the antibody according to the invention binds to a sequence consisting to SEQ ID NO:2. In some embodiments, the antibody of the invention binds to an epitope comprising at least 7 amino acid residues from SEQ ID NO:1, or from a sequence sharing at least 90% of identity over SEQ ID NO: 1.

In one embodiment, the antibody of the invention binds to an epitope comprising the amino acid sequence as set forth in SEQ ID NO: 1 or an amino acid sequence sharing at least 90% of identity over SEQ ID NO: 1.

In a particular embodiment, the antibody according to the invention wherein the antibody recognizes an epitope comprising 7 amino acid residues from SEQ ID NO:1, or from a sequence sharing at least 90% of identity over SEQ ID NO: 1.

In a particular embodiment, the antibody according to the invention wherein the antibody binds to an epitope comprising the amino acid sequence as set forth in SEQ ID NO: 1 or an amino acid sequence sharing at least 90% of identity over SEQ ID NO: 1.

In a second aspect, the invention relates to use of the antibody according to the invention for determining the level of Helix-III in a biological sample.

The level of Helix-III peptide as defined above may be determined for example by capillary electrophoresis-mass spectroscopy technique (CE-MS), flow cytometry, mass cytometry or immunoassay such as an enzyme-linked immunosorbent assay (ELISA), performed on the sample.

In some embodiments, the level of Helix-III peptide is determined by immunoassay.

As used herein, the term “Immunoassays” encompass any assay wherein a capture reagent (i.e binding partner) is immobilised on a support and wherein detection of an analyte of interest (i.e Helix-III peptide) is performed through the use of antibodies directed against the said analyte of interest (i.e Helix-III peptide). Such assays include, but are not limited to agglutination tests; enzyme-labeled and mediated immunoassays, such as enzyme-linked immunosorbent assays (ELISAs); biotin/avidin type assays; radioimmunoassays; immunoelectrophoresis; immunoprecipitation, capillary electrophoresis-mass spectroscopy technique (CE-MS) etc. The reactions generally include revealing labels such as fluorescent, chemiluminescent, radioactive, enzymatic labels or dye molecules, or other methods for detecting the formation of a complex between the antigen and the antibody or antibodies reacted therewith. Immunoassays includes competition, direct reaction, or sandwich type assays.

Typically, the antibody against Helix-III peptide is labelled with a detectable molecule or substance, such as a fluorescent molecule, a radioactive molecule or any other labels known in the art. Labels are known in the art that generally provide (either directly or indirectly) a signal. As used herein, the term “labelled”, with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance. An antibody or aptamer may be labelled with a radioactive molecule by any method known in the art. For example, radioactive molecules include but are not limited radioactive atom for scintigraphic studies such as I<123>, I<124>, In<111>, Re<186>, Re<188>. Preferably, the antibodies against HELIX-III peptide are already conjugated to a fluorophore (e.g. FITC-conjugated and/or PE-conjugated).

In a particular embodiment, the antibody according to the invention which is conjugated with a detectable label.

In a particular embodiment, the antibody according to the invention wherein the detectable label is a radioisotope, a fluorescent label, a chemiluminescent label, an enzyme label, or a bio luminescent label.

In a particular embodiment, the antibody according to the invention wherein the label is selected from the group consisting of 0-galactosidase, glucose oxidase, peroxidase (e.g. horseradish peroxidase) and alkaline phosphatase.

In some embodiments, the level of Helix-III peptide is determined by enzyme-labeled and mediated immunoassays (ELISA).

In some embodiments, the level of Helix-III peptide is determined by direct ELISA. The HELIX-III peptide is directly immobilized to a surface of a multi-well plate and detected with a biotin-conjugated detection antibody specific for the HELIX-III peptide. This antibody is directly conjugated to a detection system (horseradish peroxidase (HRP)-conjugated Streptavidin or other detection molecules).

In some embodiments, the level of Helix-III peptide is determined by indirect ELISA. The HELIX-III peptide is directly immobilized to a surface of a multi-well plate and detected with an unconjugated primary detection antibody specific for the Helix-III peptide. A conjugated secondary antibody directed against the host species of the primary antibody is then added. Substrate then produces a signal proportional to the amount of Helix-III peptide bound in the well.

In some embodiments, the level of Helix-III peptide is determined by sandwich ELISA.

According to the invention, “sandwich” ELISA refers to an immunoassay wherein free Helix-III peptide may be sandwiched between two antibodies that specifically bind to free Helix-III peptide. Typically, the Helix-III peptide is conjugated with a detection system (such as horseradish peroxidase (HRP)-conjugated Streptavidin or other detection molecules).

In another embodiment, Helix-III peptide is identified by immunohistochemistry. Typically, an immunohistochemistry of biological obtained from a subject is performed by using a specific antibody anti Helix-III peptide. In a particular embodiment, the antibody is a polyclonal antibody against Helix-III peptide.

In a particular embodiment, the antibody is a monoclonal antibody against Helix-III peptide.

In a third aspect, the invention relates to an in vitro method for diagnosing a collagen degradation associated disease in a subject comprising the steps of:
i) contacting a biological sample with the antibody of claim 1 under conditions that allow an immunocomplex of the Helix-III peptide and antibody,
ii) detecting and/or quantifying the level of Helix-III peptide in said biological sample, and
iii) concluding that the subject suffers from a collagen degradation associated disease when the level of Helix-III peptide determined at step ii) is higher than the reference value or concluding that the subject does not suffer from a collagen degradation associated disease when the level of Helix-III peptide determined at step ii) is lower than the reference value.

As used herein term “diagnosing” refers to classifying a disease or a symptom, determining a severity of the disease, monitoring disease progression, forecasting an outcome of a disease and/or prospects of recovery. The present invention relates to a method for diagnosing rheumatoid arthritis (RA).

As used herein, the term “collagen degradation associated disease” refers to diseases caused by an abnormal collagen degradation. In particular, the collagen degradation associated disease is selected but not limited to: osteoporosis, Paget's disease, bone tumours, drug-related bone loss, osteoarthritis, rheumatoid arthritis, juvenile arthritis, psoriatic arthritis, spondyloarthropathies, fibrosis.

In a particular embodiment, the collagen degradation associated disease is rheumatoid arthritis (RA).

As used herein, the term “rheumatoid arthritis (RA)” refers to a chronic autoimmune disease and characterized by inflammation and cellular proliferation in the synovial lining of joints that can ultimately result in cartilage and bone destruction, joint deformity and loss of mobility. RA usually causes problems in several joints at the same time, often in a symmetric manner. Early RA tends to affect the smaller joints first, such as the joints in the wrists, hands, ankles and feet. As the disease progresses, joints of the shoulders, elbows, knees, hips, jaw and neck can also become involved. Unlike other arthritic conditions that only affect areas in or around joints, RA is a systemic disease which can cause inflammation in extra-articular tissues throughout the body including the skin, blood vessels, heart, lungs and muscles.

In another embodiment, the collagen degradation associated disease is fibrosis.

As used herein, the term “fibrosis” refers to the formation of fibrous tissue as a reparative or reactive process, rather than as a normal constituent of an organ or tissue. Fibrosis is characterized by myofibroblast accumulation and collagen deposition in excess of normal deposition in any particular tissue. The term is used synonymously with “myofibroblast accumulation and collagen deposition”. In some embodiments, the fibrosis affects at least one organ selected from the group consisting of skin, eye, intestine, heart, liver, lung, and kidney. Examples of fibrosis include, without limitation, dermal scar formation, keloids, liver fibrosis, lung fibrosis, kidney fibrosis, glomerulosclerosis, pulmonary fibrosis (e.g. idiopathic pulmonary fibrosis), liver fibrosis (e.g. following liver transplantation, liver fibrosis following chronic hepatitis C virus infection), renal fibrosis, intestinal fibrosis, interstitial fibrosis, cystic fibrosis of the pancreas and lungs, injection fibrosis, endomyocardial fibrosis, mediastinal fibrosis, myelofibrosis, retroperitoneal fibrosis, progressive massive fibrosis, nephrogenic systemic fibrosis.

In a particular embodiment, the collagen degradation associated disease is hepatic fibrosis.

As used herein, the term “liver fibrosis” or “hepatic fibrosis” refers to the wound-healing response to chronic liver injury. Liver fibrosis is characterized by the accumulation of extracellular matrix that can be distinguished qualitatively from that in normal liver. Left unchecked, hepatic fibrosis progresses to cirrhosis (defined by the presence of encapsulated nodules), liver and organ failure, and death. Chronic liver injury may be the result of chronic alcohol consumption (alcoholic liver disease, steatohepatitis (ASH)), overfeeding, insulin resistance, type 2 diabetes (non-alcoholic fatty liver disease, NASH, steatosis), idiopathic portal hypertension, hepatic fibrosis (including congenital hepatic fibrosis), autoimmune hepatitis, primary sclerosing cholangitis, or primary biliary cirrhosis. In some embodiments, the fibrosis is associated with liver steatosis.

As used herein, the term “subject” refers to any mammals, such as a rodent, a feline, a canine, and a primate. Particularly, in the present invention, the subject is a human. In a particular embodiment, the subject is a human who is susceptible to have a collagen degradation associated disease. In a particular embodiment, the subject is a human who is susceptible to have RA. In a particular embodiment, the subject is a human who is susceptible to have fibrosis such as hepatic fibrosis.

As used herein, the term “biological sample” refers to a sample obtained from a subject, for example blood, saliva, breast milk, urine, semen, bronchoalveolar lavage, blood plasma, synovial fluid or serum. In a particular embodiment, the biological sample is serum sample. In a particular embodiment, the biological sample is synovial fluid.

As used herein, the term “reference value” refers to a threshold value or a cut-off value.

Typically, a “threshold value” or “cut-off value” can be determined experimentally, empirically, or theoretically. A threshold value can also be arbitrarily selected based upon the existing experimental and/or clinical conditions, as would be recognised by a person of ordinary skilled in the art. For example, retrospective measurement of the Helix-III peptide in properly banked historical subject samples may be used in establishing the reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. The full name of ROC curve is receiver operator characteristic curve, which is also known as receiver operation characteristic curve. It is mainly used for clinical biochemical diagnostic or prognostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER. SAS, CREATE-ROC.SAS, GB STAT VI0.0 (Dynamic Microsystems, Inc. Silver Spring, Md., USA), Stata/Se version 12.0 software (StataCorp LP, College Station, TX, USA), etc.

In some embodiments, the reference value is the level of Helix-III peptide in a healthy subject (i.e that has not been diagnosed for an AR disorder).

In some embodiments, the reference value is the Helix-III peptide detected in previous samples obtained from the subject. In some embodiment, the reference value is the upper limit of values determined in healthy subjects (i.e that has not been diagnosed for a collagen degradation associated disease and other musculoskeletal disease such as osteoarthritis, spondolyloarthropathies, osteoporosis.

In some embodiments, the reference value is ranging from 12 to 73 ng/ml.

The Helix-III ELISA method as defined above is useful for the clinical investigation of patients with a collagen degradation associated disease and for identifying collagen degradation associated disease patients at higher risk of progression.

Accordingly, in a fourth aspect, the invention relates to an in vitro method for monitoring collagen degradation associated disease progression in a subject comprising the steps of:
i) contacting a biological sample obtained from said subject with an antibody directed against the Helix-III peptide according to the invention,
ii) detecting and/or quantifying the level of Helix-III peptide in said biological sample, and
iii) concluding that the subject has a risk of collagen degradation associated disease progression when the level of Helix-III peptide determined at step ii) is higher than the reference value or concluding that the subject has a low risk of collagen degradation associated disease progression when the level of Helix-III peptide determined at step ii) is lower than the reference value.

In some embodiment, the in vitro method for monitoring collagen degradation associated disease progression in a subject comprises a further step of detecting and/or quantifying bone erosion in said subject.

Accordingly, in some embodiment, the invention relates to an in vitro method for monitoring collagen degradation associated disease progression in a subject comprising the steps of:
i) contacting a biological sample obtained from said subject with an antibody directed against the Helix-III peptide according to the invention,
ii) detecting and/or quantifying the level of Helix-III peptide in said biological sample,
iii) detecting and/or quantifying bone erosion in said subject, and
iv) concluding that the subject has a risk of collagen degradation associated disease progression when the level of Helix-III peptide determined at step ii) and the level of bone erosion determined at step iii) are higher than the reference values or concluding that the subject has a low risk of collagen degradation associated disease progression when the level of Helix-III peptide determined at step ii) and the level of bone erosion determined at step iii) are lower than the reference values.

By “predicting the progression and/or monitoring the progression” it is meant in the invention that the method allows to predict the likely outcome of collagen degradation associated disease such as RA or fibrosis. More particularly, the prognosis method can evaluate the survival rate, said survival rate indicating the percentage of people, in a study, who are alive for a given period of time, after diagnosis of collagen degradation associated disease such as RA or fibrosis. Prognosis can also evaluate the risk of progression of joint damage evaluated by changes in radiological scores, such as the Sharp score, or magnetic resonance image scores. This information allows the practitioner to determine if a medication is appropriated, and in the affirmative, what type of medication is more appropriate for the subject. The prediction of progression and/or the monitoring of the progression of collagen degradation associated disease such as RA or fibrosis is realised by measuring the level of Helix-III in serum sample by ELISA or identifying the level of Helix-III by IHC. An increase level of the level of Helix-III in serum sample in the subject is associated with a high risk of having RA progression, i.e. a bad prognosis of collagen degradation associated disease such as RA or fibrosis.

As used herein, the term “bone erosion” refers to a peri-inflammatory destructive bone lesion. Typically, bone erosion can be diagnosed and quantified by a radiographic evaluation or Magnetic Resonance Imaging (MRI). As example, radiographs of the hands, wrists or feet in a posteroanterior view can be taken. The resulting images can be scored according to the van der Heijde modified Sharp Score (mSS), or alternative scoring methods, in order to establish an erosion score.

The method according to the invention is suitable for identifying subjects with a higher risk of rapid progression who escape from treatment, in order to adapt the therapy.

Accordingly, in a fifth aspect, the invention relates to an in vitro method for predicting whether a subject will achieve a response to a collagen degradation associated disease treatment in a subject comprising the steps of:
i) contacting a biological sample obtained from said subject with an antibody directed against the Helix-III peptide,
ii) detecting and/or quantifying the level of Helix-III peptide in said biological sample, and
iii) concluding that the subject will not achieve a response to said treatment when the level of Helix-III peptide determined at step ii) is higher than the reference value or concluding that the subject will achieve a response to said treatment when the level of Helix-III peptide determined at step ii) is lower than the reference value.

As used herein, the term “predicting” means that the subject to be analyzed by the method of the invention is allocated either into the group of subjects who will respond, or into a group of subjects who will not respond to a treatment.

The method according to the invention is suitable to predict the risk of relapse to treatment in a subject suffering from collagen degradation associated disease such as RA or fibrosis. Accordingly, the invention relates to an in vitro method for predicting the risk of relapse to treatment in a subject suffering from collagen degradation associated disease such as RA or fibrosis comprising the steps of.
i) contacting a biological sample obtained from said subject with an antibody directed against the Helix-III peptide,
ii) detecting and/or quantifying the level of Helix-III peptide in said biological sample, and
iii) concluding that the subject is at risk of relapse to said treatment when the level of Helix-III peptide determined at step ii) is higher than the reference value or concluding that the subject is not at risk of relapse to said treatment when the level of Helix-III peptide determined at step ii) is lower than the reference value.

As used herein, the term “risk” in the context of the present invention, relates to the probability that an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

As used herein, the terms “will achieve a response” or “respond” refer to the response to a treatment of the subject suffering from collagen degradation associated disease such as RA or fibrosis. Typically, such treatment induces, ameliorates or otherwise causes an improvement in the pathological symptoms, disease progression or physiological conditions associated with or resistance to succumbing to collagen degradation associated disease such as RA or fibrosis. In particular, in the context of the invention, the term “respond” refers to the ability of corticosteroid treatment to an improvement of the pathological symptoms, thus, the subject presents a clinical improvement compared to the subject who does not receive the treatment. The said subject is considered as a “responder” to the treatment. The term “not respond” refers to a subject who does not present any clinical improvement to the treatment with RA treatment. This subject is considered as a “non-responder” to the treatment. Accordingly, the subject as considered “non-responder” has a particular monitoring in the therapeutic regimen. In a particular embodiment, the response to a treatment is determined by the clinical DAS or ACR scores. This criterion refers to a set of published rules that define when the disease in the subjects improve (“respond”), stay the same (“stabilize”), or worsen (“progress”) during treatment.

As used herein, the term “relapse” refers to the return of signs and symptoms of a disease after a subject has enjoyed a remission after a treatment. Thus, if initially the target disease is alleviated or healed, or progression of the disease was halted or slowed down, and subsequently the disease or one or more characteristics of the disease return, the subject is referred to as being “relapsed.”

As used herein, the term “treatment of collagen degradation associated disease such as RA or fibrosis” refers to medications, occupational or physical therapy.

In a particular embodiment, the treatment of collagen degradation associated disease such as RA or fibrosis is selected but not limited to:
i) NSAIDs: Nonsteroidal anti-inflammatory drugs (NSAIDs) can relieve pain and reduce inflammation. Over-the-counter NSAIDs include but not limited to ibuprofen (Advil, Motrin IB, others) and naproxen sodium (Aleve);
ii) Corticosteroid: Corticosteroid drugs used in the treatment of RA typically include prednisone and prednisolone;
iii) Conventional disease-modifying antirheumatic drugs (DMARDs). These drugs can slow the progression of rheumatoid arthritis. Common DMARDs include methotrexate (Trexall, Otrexup, others), leflunomide (Arava), hydroxychloroquine (Plaquenil) and sulfasalazine (Azulfidine);
iv) Biologic agents. Also known as biologic response modifiers, this newer class of DMARDs includes abatacept (Orencia), adalimumab (Humira), anakinra (Kineret), certolizumab (Cimzia), etanercept (Enbrel), golimumab (Simponi), infliximab (Remicade), rituximab (Rituxan), sarilumab (Kevzara) and tocilizumab (Actemra);
v) Targeted synthetic DMARDs. Baricitinib (Olumiant), tofacitinib (Xeljanz), upadacitinib (Rinvoq) and filgotinib (Jyseleca);
vi) Amiodarone, chlorpromazine, tolbutamide, isoniazid, methyldopa, oxyphenisatine.

In a sixth aspect, the invention relates to a kit for use in the method according to the invention, wherein said kit comprising:
a solid support,
a binding partner against Helix-III peptide, and
instructions for use.

In a particular embodiment, the binding partner is directed against the Helix-III peptide.

In some embodiments, the binding partner is an antibody or aptamer. In a particular embodiment, the binding partner is an antibody polyclonal against Helix-III peptide. In a particular embodiment, the binding partner is an antibody monoclonal directed against Helix-III peptide. In some embodiments, the binding partner is labelled with a detectable molecule or substance, such as a fluorescent molecule, a radioactive molecule or any other labels known in the art. Labels are known in the art that generally provide (either directly or indirectly) a signal. As used herein, the term “labelled”, with regard to the antibody or aptamer, is intended to encompass direct labelling of the antibody or aptamer by coupling (i.e., physically linking) a detectable substance, such as a radioactive agent or a fluorophore (e.g. fluorescein isothiocyanate (FITC) or phycoerythrin (PE) or Indocyanine (Cy5)) to the antibody or aptamer, as well as indirect labelling of the probe or antibody by reactivity with a detectable substance.

In one embodiment, the capture anti-Helix-III antibody is coated directly or indirectly to a solid support, said solid support comprising a protein binding surface such as high-binding well ELISA plates; nitrocellulose (e. g., in membrane or microtiter well form); polyvinylchloride (e. g., sheets or microtiter wells); polystyrene latex (e.g., beads or microtiter plates); polyvinylidine fluoride; diazotized paper; nylon membranes; activated beads, magnetically responsive beads, and the like.

The invention also relates to a computer program product comprising code instructions for implementing any of the above methods for diagnosing, monitoring the collagen degradation disease progression and predicting the response to a treatment.

Accordingly, in seventh aspect, the invention relates to a computer-implemented method for diagnosing collagen degradation associated disease in a subject, comprising the following steps:
i) contacting a biological sample with the antibody according to the invention;
ii) quantifying the level of Helix-III peptide in said biological sample;
iii) incorporating said quantified value in a software with clinical parameters; and
iv) concluding that the subject suffers from a collagen degradation associated disease when the level of Helix-III peptide associated with clinical parameters determined at step iii) is higher than the reference value or concluding that the subject does not suffer from a collagen degradation associated disease when the level of Helix-III peptide associated with clinical parameters determined at step iii) is lower than the reference value.

In a particular embodiment, the level of Helix-III peptide is associated further with biological parameters and/or imaging scores.

As used herein, the term “clinical parameters” refers to age, sex, disease duration, swollen joint count (SJC), tender joint count (TJC), erythrocyte sedimentation rate (ESR), C-reactive protein (CRP), Disease Activity Score (DSA), anti-citrullinated protein antibody (ACPA), radiological scores, such as the Sharp score, or magnetic resonance image scores.

The invention will be further illustrated by the following figures and examples. However, these examples and figures should not be interpreted in any way as limiting the scope of the present invention.

### FIGURES

FIG. 1: Example of HELIX-III ELISA calibration curve. A log-lin-4-parameter calibration curve is used to analyse the S-HELIX-III assay results. The HELIX-III concentration (ng/ml) of each sample is determined by interpolation from the standard curve. Four Parameter Logistic Equation: y=b+(a−b)/(1+xc)\hat{ }d, a=1.919, b=0.059, c=0.081, d=0.952, R2 Correlation Coefficient=0.999.

FIG. 2: Helix-III stability test in serum samples stored at 4° C. or room temperature (RT) and subjected to successive freeze/thaw cycles

FIG. 3: The graph shows the competitive inhibition of the ELISA using Helix-III synthetic peptide (PPGPPGPhypGTS—SEQ ID NO:2) used as standard with:
Helix-III peptide sequence in which hydroxyproline (hyp) was replaced by a proline (PPGPPGPPGTS—SEQ ID NO:3),
Helix-III peptide extended by 1 amino acid (glycine) (PPGPPGPhypGTSG—SEQ ID NO:5) or shortened by 1 amino acid at the C-terminal end (PPGPPGPhypGT—SEQ ID NO:4),
an analogous sequence of human type IV collagen (LQGPPGPPGTS—SEQ ID NO:7) whose 9/11 amino acids were identical to Helix-III peptide at the C-terminal end
an analogous sequence of human type XI collagen (SDGAPGPPGTS—SEQ ID NO:8) whose 7/11 amino acids were identical to Helix-III peptide at the C-terminal end
an analogous sequence of human type III collagen (PSGPPGKDGTS—SEQ ID NO:6) whose only 3 amino acids at the C-terminal end were identical to Helix-III peptide.
The y-axis shows the relative binding of/the Helix-III antibody on Helix-III-coated microtiter plates at different concentrations of each peptide (B) expressed as a percentage of the binding with no competitor peptide (BO). The x-axis shows the molar concentration of each peptide for purposes of comparison.

FIG. 4: IHC the anti-Helix III rabbit polyclonal antibody

FIG. 5: Box plot of serum Helix III levels in healthy controls and subjects with RA. The upper and lower limits of the box represent the 75 and 25 percentiles of the distribution respectively. The horizontal bar and the small square in the box are respectively the median and mean values of each group.

FIG. 6: Scatter plot of S-Helix II vs CRP in RA patients (n=51)

FIG. 7: Baseline serum levels of Helix III are increased in patients with early arthritis who had structural progression within the following 5 years. The box-plot graph represents baseline serum Helix III values in patients with either no structural progression (<5 points increase of van der Heijde modified total Sharp score between baseline and 5 years; left) or with progression (>5 points increase in the radiographic score) within the subsequent 5 years. The upper and lower limits of the box represent the 75 and 25 percentiles of the distribution, respectively. The horizontal bar in the box is the median value of each group.

FIG. 8: Association of baseline serum Helix III and classical risk factors with progression of joint damage in patients with early arthritis. The graph shows the odds-ratio of 5-year radiologic progression of total damage adjusted for gender, BMI and diagnosis of RA in patients with levels of quantitative variables (serum Helix III, DAS28, CRP) in the highest quintile or ACPA positive compared to subjects with values in the 4 lowest quintiles or ACPA negative, respectively.

FIG. 9: Combination of serum Helix III with bone erosion to improve the prediction of progression in patients with early arthritis. The bars represent the odds-ratio of total joint damage 5-year progression in patients with baseline levels of serum Helix III in the highest quintile (High Helix III) compared to subjects with levels in the 4 lowest quintiles, in patients with bone erosion vs patients with no erosion and in patients with both high Helix III and bone erosion compared to all the other subjects. On each bar the OR (95% CI), adjusted for gender, BMI and diagnosis of RA, is indicated.

### EXAMPLE 1

### Material & Methods

### Immunoassay for Helix-III Peptide.

BSA, PBS, TBS and Tween20 were purchased from Euromedex (Strasbourg, France). Synthetic peptides, including biotinylated and keyhole limpet hemocyanin (KLH)-coupled peptides, were synthesized to >90% purity by ProteoGenix SAS (Schiltigheim, France).

Production of polyclonal antibodies against HELIX-IT Free Synthetic Helix III peptide 178-PPGPPGPhypGTS-188 (SEQ ID NO:2) (where hyp is hydroxyproline) derived from the sequence of the α_1 chain of human type III collagen (HELIX-III; SwissProt accession no. P02461) were synthesized to >95% purity by Proteogenix SAS (Schiltigheim, France). Biotinylated and keyhole limpet hemocyanin (KLH)—coupled Helix-III peptides were also synthesized. 2 Rabbits were injected intraperitoneally with 0.5 mg/rabbit of KLH conjugated peptide in Complete Freund's Adjuvant. Immunizations were repeated 6 times during 56 days with 250 μg/rabbit of immunogen emulsified in Freund's incomplete adjuvant. At each bleeding, antiserum was screened by titration for the presence of anti-HELIX-III antibodies. Titration was performed by investigating the binding of subsequent dilutions of the antiserum on microtiter plates coated with biotinylated HELIX-III peptide (see below). The titer was defined as the dilution of the antiserum giving 50% of the absorbance of the undiluted antiserum. The antisera with the highest titers were selected for the development of the ELISA.

### Helix-III ELISA

Biotinylated HELIX-III peptide diluted in PBS with 0.1 gm/liter of BSA at pH 7.4 (200 μl of Biotinylated HELIX-III peptide at 0.75 μg/liter) was pipetted into each well of a Nunc Immobilizer Streptavidin plate (Thermo Fisher Scientific Inc., France). The plate was incubated for 2 hours at room temperature. The plate was then washed 5 times with a washing buffer made up of TBS with 0.5 gm/liter of BSA and 0.05% (vo/vol) of Tween 20, pH 7.2. 50 μl of calibrator, or control, or unknown serum samples, prediluted to one third with TBS sample buffer containing 1 gm/liter of BSA and 0.05% (vol/vol) of Tween 20 at pH 7.2, was pipetted into each well and 50 μl of primary antibody (polyclonal antibody against HELIX-III peptide) diluted at 5.5 ng/ml in washing buffer was added into each well. After incubation for 18 hours at 4° C. under stirring, the plate was washed 5 times with washing buffer, and 100 μl (100 μg/liter) of a solution of peroxidase-conjugated goat anti-rabbit antibody (Jackson ImmunoResearch, USA) diluted in the washing buffer was pipetted into each well. The plate was incubated for 1 hour at room temperature. After incubation, the wells were washed 5 times with washing buffer and 100 μl H2O2/tetramethylbenzidine substrate solution (Interchim, France) was added by well. After incubation at room temperature for around 20 minutes in dark, the colorimetric reaction was stopped by the addition of 100 μl of 0.5M H2SO4, and the optical density at 450 nm corrected for the absorbance at 620 nm was measured. All samples were measured in duplicate.

### Statistical Analysis

All data are expressed as the mean±SD unless otherwise specified. Between-group comparisons were performed using the Kruskal-Wallis nonparametric test or the nonparametric Mann-Whitney rank test or parametric student t-test after log conversion. Correlations were estimated by nonparametric Spearman's rank correlation coefficient. All statistical analyses were carried out using Xcel Stat software.

### IHC Analyses

Synovium biopsies were recovered immediately after surgery in sterile conditions. All biopsies were fixed in 4% Paraformaldehyde solution for 48 h at room temperature (RT). They were then processed according to standard pathological procedures, embedded in paraffin and cut in section of 5 μm. Sections were deparaffinized in Methylcyclohexane and rehydrated with a succession of alcohol bath containing increasing water percentage. After the last bath in 100% waters, serial cut section from each patient biopsy was either stained with a standard Haematoxylin Eosin coloration or either use for HIC with specific antibodies. HIC was carried out with Polink-TRP plus Rabbit AEC Detection System (GBI Labs Mukilteo, WA, USA). Deparaffinized sections were treated with Endogen Peroxidase Blocking Reagent (3% H2O2) for 10 min, demasked by incubation for 2 h at 80° C. in citrate buffer (pH 6.0) and blocked with the Protein Block Reagent. Sections were then incubated overnight h at 4° C. with IgG Rabbit antibody against Helix-III, or with IgG Rabbit anti human type III collagen antibody (gift from NOVOTEC SAS, Bron, France), or with Isotype control Rabbit IgG (Abcam Cambridge, UK) diluted at a same concentration of 0.45 and 0.9 μg/ml. This incubation was followed by a 30-min incubation with Rabbit Antibody Enhancer and then with Polymer horseradish peroxidase (HRP) conjugated for Rabbit during 30 min. Development was performed using 3-Amino-9-Ethylcarbazole (AEC) chromogen. Finally, the sections were counter-stained with Mayer's acidic hematoxylin, rinced in water, and mounted in GB-Mount. Digital pictures were obtained using a computer assisted microscope and only modified by enhancing the contrast and light; no background was removed or altered.

### Results

### Analytic Performance of HELIX-III ELISA

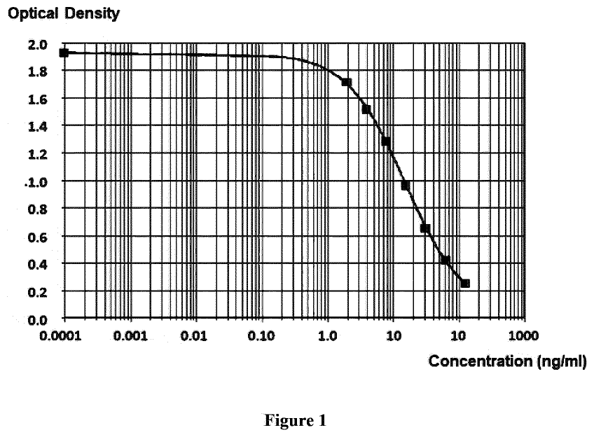

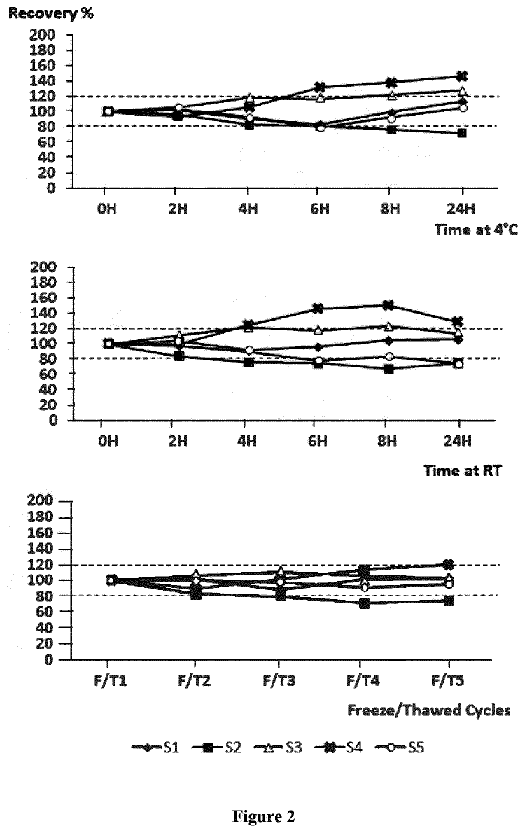

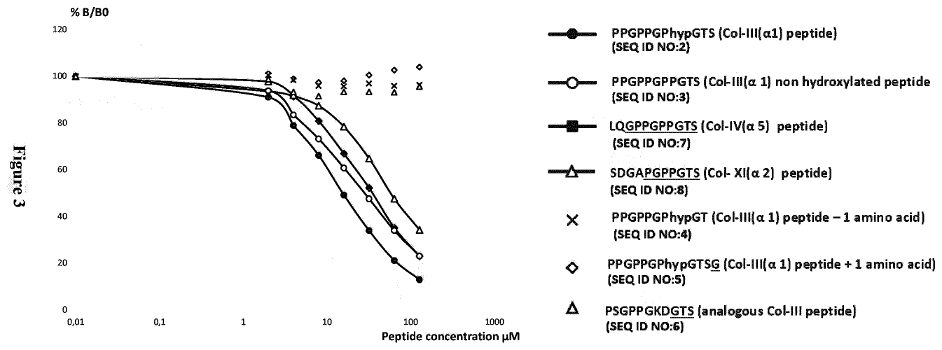

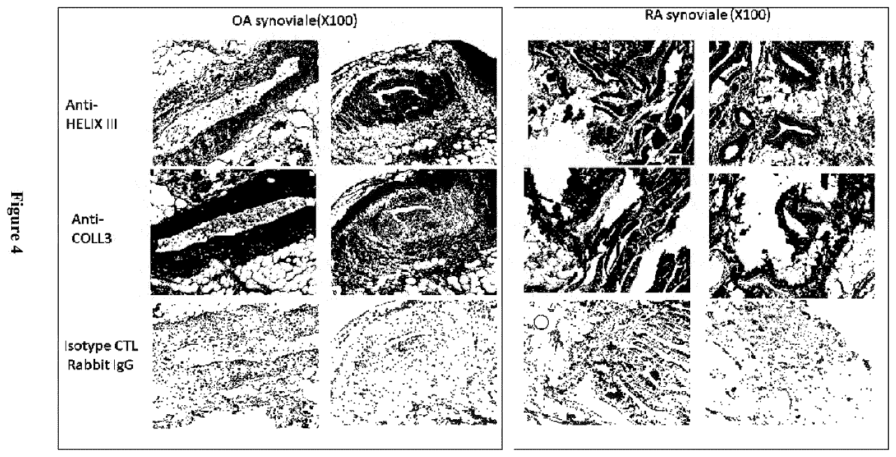

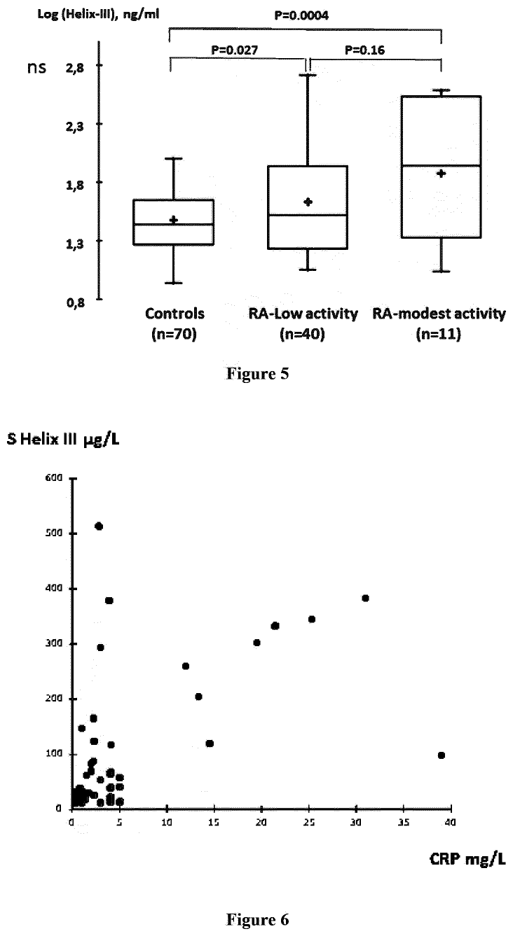

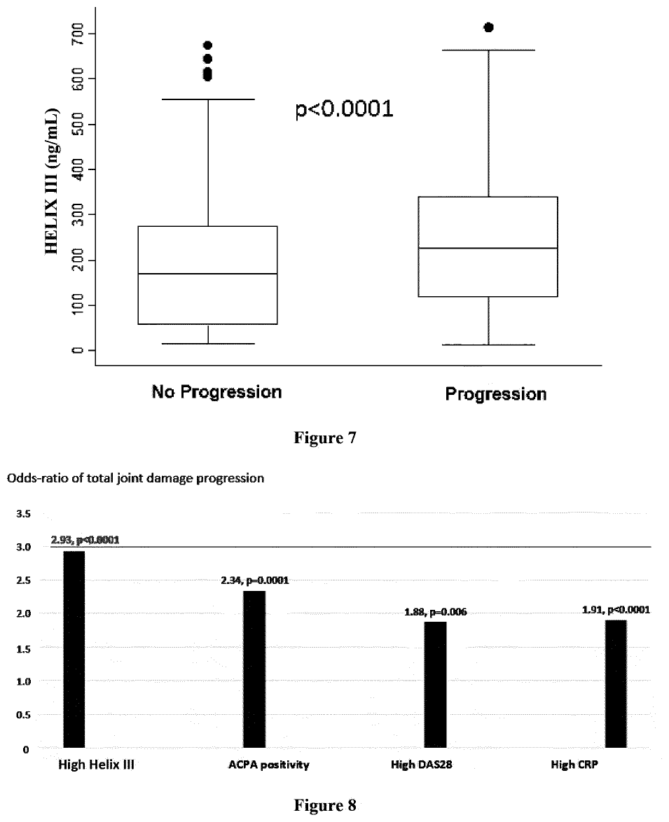

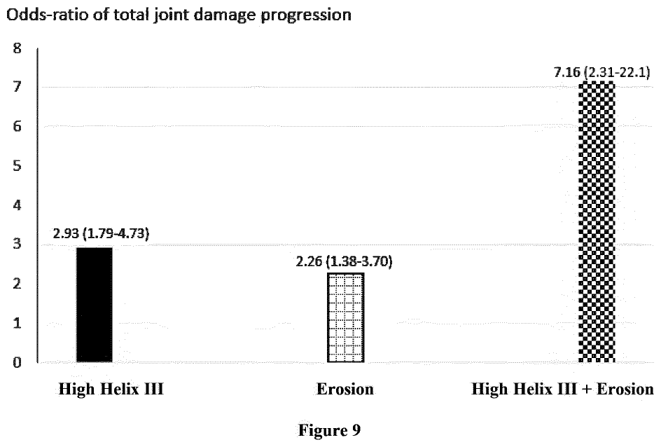

We have developed a competitive ELISA using a polyclonal antibody directed against the HELIX-III sequence. As shown in FIG. 1 and Table 1, the calibration curve was ranged between 0 and 128 ng/ml. The inter-assay variation of the calibrators was between 2.3 and 7.1% with a standard precision between 93 and 103%. Serum samples require a ⅓ predilution before assaying. The detection limit, defined as the concentration corresponding to 3 SD above the mean of 20 determinations of the zero calibrator, was determined to be 0.21 ng/ml. the Lower Limit of Quantification was assessed as being 2.55 ng/ml which corresponds to the closest value above the first standard with an acceptable coefficient of variation percentage. The upper limit of quantitation has been determined as being 95 ng/ml without the predilution factor. It corresponds to 285 ng/ml in serum samples according to predilution factor. The samples intraassay variation was assessed by 16 measurements of 5 different serum samples (mean levels of 5.8, 14.4, 38.2, 46.0 and 105.6 ng/ml) in the same run ranged from 5.1% to 9.7%. The sample interassay variation was determined by measurements of 5 different serum samples (mean levels of 6.4, 15.1, 41.5, 51.2 and 102.8 ng/ml) in 16 different runs. The interassay variation was ranged from 8.1% to 14.1%. The recovery of the dilution was determined using 8 different serum samples (initially diluted to ⅓ in Sample Buffer) then diluted again to ½ in Sample Buffer (Table 2). The recovery percentages range from 80 to 108%. The Spiking recovery was determined by addition of known quantities of HELIX-III peptide (20-40 ng/ml) into 3 different serum samples (Table 3). The percent recovery was range of 88 to 125% (mean 104±14). Serum HELIX-III levels remained stable for at least 6 hours of incubation at 4° C. and 4 hours at room temperature with a recovery between 80-120% relative to serum not incubated. Beyond these incubation times the variability of serum Helix-III levels increased. Likewise, 4 repeated freeze-thaw cycles did not substantially change the concentration of HELIX-III for most of the serum. (FIG. 2). The specificity of the antibody was investigated by experiments involving competitive inhibition between HELIX-III peptide and different synthetic peptides. As shown in FIG. 2, there was no significant crossreactivity of the antibody with the HELIX-III peptides that were shortened (PPGPPGPhypGT=SEQ ID NO:4) or extended (PPGPPGPhypGTSG=SEQ ID NO:5) by 1 amino acid at the C-terminal end up to a concentration of 0.15 μM. The antibody did not demonstrate significant immunoreactivity with the peptide PSGPPGKDGTS (SEQ ID NO: 6) which is analogous sequence of human type III collagen close to that of Helix-III. The antibody recognized but a less extend the HELIX-III sequence in which the hydroxyproline has been replaced by a proline. The HELIX-II sequence was also recognized but with a much lower affinity (a 10-fold higher amount of HELIX-II peptide is needed to obtain a comparable shift in the assay) (FIG. 3). In summary, to be immunoreactive in the HELIX-III ELISA, the collagen fragments need to have a free serine residue at the C-terminal end. The hydroxylation of the proline residue in the sequence does not appear to be essential for recognition. More than 4 amino acids at the C-terminal end identical to those of the HELIX-III sequence are also necessary for recognition, but do not provide full immunoreactivity.

Immuno Staining with Anti-Helix-III Antibody of Synovial Tissues of Patients with OA and RA

To investigate whether the Helix III Peptide is also present in vivo, immunohistochemistry of human synovial of patients with OA or RA was performed using the specific polyclonal antibody that was generated or the immunoassay. As shown in FIG. 3 and FIG. 4, HELIX III peptide was detected in synovium of OA and RA patients specifically in the synovial interstitial tissue and around vessels.

### Serum HELIX-III Levels in Normal Population of Men and Women

Serum Helix-III levels were measured in a middle-age normal population. No significant variation of HELIX-III was observed according to gender (Table 4).

Serum HELIX-III Levels in Healthy Controls and Patients with Rheumatoid Arthritis

Table 5 shows the characteristics of the RA population and biological serum values of CRP and ESR. As shown in the Table 5 and the FIG. 5, median serum HELIX-III levels were significantly higher in patients with moderate (p=0027) and active RA (p=00004) compared with those in age-matched controls. When all RA patients were analysed together, there was a significant correlation between serum HELIX-III levels and C Reactive Protein (CRP) and erythrocyte sedimentation rate (ESR) (r=0.59, P<0.0001 and r=0.44, P=0.001 respectively) FIG. 6 and Table 6.

TABLE 1
The Inter assay variations of calibrators
were determined on 16 different runs.

| | | | | | | | |
|---|---|---|---|---|---|---|---|
| Calibrator | 2 | 4 | 8 | 16 | 32 | 64 | 128 |
| (ng/ml) | | | | | | | |
| Measured | 1.86 | 4.13 | 7.93 | 16.3 | 31.8 | 62.4 | 132.3 |
| values (ng/ml) | | | | | | | |
| CV (%) | 7.1 | 3.3 | 4.0 | 2.3 | 2.5 | 3.8 | 3.5 |
| Accuracy (%) | 93 | 103 | 99 | 102 | 99 | 98 | 103 |

TABLE 2
Dilution of 8 Serum Samples in HELIX-III immunoassay

| | | | % |
|---|---|---|---|
| Sample | Dilution | S-HELIX-III ng/ml | Recovery |
| S1 | Initial | 21.37 | — |
| | dilution | | |
| | ½ | 10.34 | 97 |
| S2 | Initial | 8.14 | — |
| | dilution | | |
| | ½ | 4.30 | 106 |
| S3 | Initial | 27.19 | — |
| | dilution | | |
| | ½ | 13.44 | 99 |
| S4 | Initial | 16.63 | — |
| | dilution | | |
| | ½ | 6.69 | 80 |
| S5 | Initial | 23.04 | — |
| | dilution | | |
| | ½ | 11.44 | 99 |
| S6 | Initial | 22.54 | — |
| | dilution | | |
| | ½ | 12.21 | 108 |
| S7 | Initial | 17.81 | — |
| | dilution | | |
| | ½ | 8.45 | — |
| S8 | Initial | 11.97 | |
| | dilution | | |
| | ½ | 5.20 | 87 |

TABLE 3
Serum Spiking Recovery of 3 Serum Samples in HELIX-III
immunoassay

| | Sample | Added | Expected | Assay | |
|---|---|---|---|---|---|
| | Values | Values | Values | Values | Recovery |
| | ng/ml | ng/ml | ng/ml | ng/ml | % |
| R1 | 17.35 | 40 | 57.35 | 50.55 | 88 |
| | | 20 | 37.35 | 40.32 | 108 |
| R2 | 31.62 | 40 | 71.62 | 77.08 | 108 |
| | | 20 | 51.62 | 64.75 | 125 |
| R3 | 19.62 | 40 | 59.62 | 54.13 | 91 |
| | | 20 | 39.62 | 41.45 | 105 |
| | | | | Mean | 104 |

TABLE 4
Demographics, DAS 28, CRP and Helix III data in controls and RA subjects (BMA and SNA studies)

| | | Age | BMI | Disease | | CRP | Sed Rate |
|---|---|---|---|---|---|---|---|
| | Gender(F/M) | (year) | (Kg/m2) | duration (year) | DAS 28 | (mg/L) | (mm) |
| Controls | 53/17 | 59.1 ± 11.1 | 23.9 ± 4.3 | | | | |
| (n = 70) | | | | | | | |
| RA- | 33/7 | 57.7 ± 8.5 | 26.8 ± 6.1 | 16 ± 11 | 2.0 ± 0.7 | 4.1 ± 6.8 | 8.5 ± 6.9 |
| medium | | | | (med: 16 | med: 2.1 | (med: 2.3 | (med: 6.5 |
| (n = 40) | | | | [1-43]) | [0.5-3.1]) | [0.3-39]) | [1.0-29.0]) |
| RA- | 7/4 | 64.6 ± 11.4 | 26.7 ± 5.9 | 16 ± 14 | 3.6 ± 1.2 | 9.2 ± 11.7 | 16.2 ± 15.8 |
| High | | | | (med: 9 | (med: 3.5 | (med: 2.2 | (med: 14.0 |
| (n = 11) | | | | [0-41]) | [1.7-5.0]) | [0.3-31]) | [1.0-51]) |
| P value | 0.12 | 0.22 | 0.013 | 0.774 | 0.0002 | 0.909 | 0.21 |

TABLE 5
Helix III levels (μg/L) in healthy controls and patients with RA

| | | | | | | | p value vs | P values vs |
|---|---|---|---|---|---|---|---|---|
| | n | Mean | SD | median | 25 pct | 75 pct | controls (Log) | RA-medium (Log) |
| Controls | 70 | 35.5 | 22.3 | 27.4 | 18.6 | 44.4 | | |
| RA- | 40 | 78.6 | 104.6 | 32.8 | 17.1 | 86.1 | 0.027 | |
| medium | | | | | | | | |
| RA- | 11 | 158.7 | 162.4 | 86.6 | 22.8 | 338 | 0.0004 | 0.16 |
| Active | | | | | | | | |

TABLE 6
Correlation of Helix III with biological and clinical indices of disease
activity in subjects with RA (BMA + SNA = N = 51)

| | Spearman Rank correlation | |
|---|---|---|
| | r Value | P Value |
| CRP | 0.59 | <0.0001 |
| Sed Rate | 0.44 | 0.001 |
| DAS28 | 0.17 | 0.25 |
| BMI | 0.14 | 0.35 |

### Conclusion

In summary, the inventors have developed an ELISA of a new molecular marker detecting a neoepitope generated from the cleavage of the α1 chain of type III collagen within its helical domain. Serum levels of this marker were significantly increased in patients with RA and is significantly associated to CRP and ESR levels.

### EXAMPLE 2

The aim of this part was to investigate whether serum Helix III, a new ELISA-based biochemical marker of synovial collagen turnover, was associated with progression of joint damage in patients with early arthritis.

### Material & Methods

Patients with Early Arthritis

The ESPOIR cohort (in French, the study and follow-up of early undifferentiated arthritis, NCT03666091) is a multicenter early arthritis cohort described in details elsewhere [17]. With approval of the Montpellier University (France) ethical committee, 16 university hospital rheumatology departments enrolled patients, covering a large part of the country. Clinical, laboratory, and imaging data were collected at baseline, then every 6 months for the first 2 years, then once a year. One biological resources center (Sarah Tubiana, Paris-Bichat) was in charge of centralizing and managing biological data collection. The inclusion criteria were the following: patients age 18-70 years provided signed informed consent, had 2 or more swollen joints, with a duration >6 weeks and <6 months, used no previous disease-modifying drugs and no steroids, and had no definite diagnosis of a disease other than RA or undifferentiated arthritis. Thus, the ESPOIR cohort consists of both early undifferentiated inflammatory arthritis and recently developed RA. At baseline, an exhaustive data collection was performed according to recommendations in early arthritis [18]. These include, but not limited to, the demographical variables age, gender, body weight and height and symptom duration, full clinical examination for the determination of the DAS28 score [19], and the biological variables CRP, anti-cyclic citrullinated peptide 2 antibodies (ACPA) tested by ELISA (DiaSorin, Antony, France; positive if >50 units/ml) and rheumatoid factor (RF) by ELISA (Menarini, France, both positive if >9 UI/ml) measured in a central laboratory (S. Martin, Immunology Department, Bichat Hospital, Paris, France). The diagnosis of RA was based on the American College of Rheumatology-European League Against Rheumatism (ACR-EULAR) 2010 criteria for RA at inclusion [20]. Data analyzed in the present study pertain to baseline, 12 months and 5 years of follow up in all the 788 subjects (representing 97% of the total ESPOIR cohort) who had serum available at baseline to measure Helix III.

### Radiographic Evaluation

Radiographs of the hands, wrists and feet in the posteroanterior view were taken for each patient at baseline, 12 months and 5 years. Images were centralized and scored according to the van der Heijde modified Sharp score (mSS) [21] by two experienced rheumatologists who were blinded to the patient's other data, knowing the chronological sequence. For each patient, an erosion score, a joint-space narrowing (JSN) score and a total radiographic score were assessed. Based on the reproducibility of the radiographic scoring, the smallest detectable (SDC) change was calculated at 1.0 mSS unit. Progression was defined by an increase from baseline in the mSS score of at least 1 point for progression at 12 months and 5 points for progression at 5 years. These cut-off values corresponding to the SCD (1 unit/year) of the radiographic assessment have been previously used in this cohort for other analyses [22].

### Serum Helix III Measurements

Serum Helix III measurements were performed at baseline with a new ELISA (INSERM, Lyon, France) which has been fully validated [16]. This assay uses an antibody directed against a 11 amino acid sequence from the helical portion of the alpha 1 chain of human type III collagen, which shares 70% homology with the corresponding sequence of the alpha 5 chain of human type IV collagen [16]. This assay detects only fragments containing a C-terminal neoepitope generated from the proteolysis of the type III and IV collagens by MMPs and not intact collagens [16]. There is no interaction of RF with the assay performance. The intra and inter assay coefficients of variation of serum Helix III measurements are lower than 10 and 15%, respectively.

### Statistical Analyses

Results are shown as mean±standard deviation (SD) and/or median for quantitative variables, as indicated. Because serum Col3-4 values were not normally distributed as it is usually observed for most clinical biochemical markers, Helix III levels were log-transformed before analyses. The comparison between groups of Helix III values were performed by Student's t-test. Correlation of serum Helix III levels with clinical, biological and radiographic variables was analyzed by linear regression analyses. The relationships between baseline serum Helix III values and radiographic progression were assessed by logistic regression with baseline demographic variables that were significantly associated with Helix III values as covariates, i.e., BMI, gender and RA diagnosis. To assess whether serum Helix III provided prognostic information independent from major classical risk factors, further logistic regression analyses were performed by including ACPA positivity, baseline DAS28 score and baseline CRP levels (in addition to BMI, gender and RA diagnosis) in the model. In logistic regression models, serum Helix III levels were considered either as a continuous variable after standardization in standard deviations (SD, after log transformation of Helix III values) or dichotomized in centiles. The strength of the association was expressed in odds-ratio (OR) and 95% confidence interval (CI). The sensitivity, specificity, and likelihood ratio (LR) for a positive result (i.e., sensitivity/[1−specificity]) for detecting patients with significant progression were also calculated. Primary analyses were performed in the whole cohort with 5-year progression data as dependent variable. Secondary analyses included the association of baseline Helix III with progression at 1 year and restricting the analysis to patients with a confirmed diagnosis of RA at inclusion.

### Results

### Baseline Characteristics and Crossectional Associations

Table 7 shows the baseline characteristics of the patients with early arthritis included in the study. Eighty two percent of patients had a confirmed diagnosis of RA at baseline. Age, gender distribution and disease activity data were those commonly found in a population of early arthritis patients with a median symptom duration of 4.9 month. Thirty nine percent of patients were ACPA positive. The radiological scores were low as expected in such populations and the levels of serum Helix III (median 189 ng/ml) were markedly elevated compared to values reported in healthy controls from France (median: 27 ng/ml) [17]. Serum Helix III values were on average higher in men than women (+25%, p<0.0001), in patients with RA compared to subjects with undifferentiated arthritis (+16%, p=0.004) and in ACPA (+21%, p<0.0001 vs ACPA negative subjects) or RF (+31<%, p=0.015 vs RF negative individuals) positive patients. Helix III levels correlated with body mass index (BMI) (r²=0.024, p<0.0001), but not with age (p=0.93). Serum Helix III levels were slightly correlated with the bone erosion score (r²=0.07, p=0.016), subjects with erosion having on average 16% (p=0.0074) higher values than those with no erosion. There was also a significant positive correlation of Helix III with DAS 28 (r²=0.14, p<0.0001) and CRP (r²=0.40, p<0.0001).

### Helix III and Progression

### 5-Year Progression

Patients with progression of total damage at 5 years had 36% higher baseline serum Helix III levels on average than patients with no progression (FIG. 7). Levels were also higher in patients with progression of erosion (+31%, p=0.0035) or JSN (+26%, p=0.001) than subjects with no progression. The association of baseline serum Helix III with progression was first investigated considering levels of the marker as a continuous variable. As shown on Table 8, after adjustment for gender, BMI and RA diagnosis, each SD increase of baseline Helix III was significantly associated with a 1.51 to 1.55 higher risk of progression. When the variables ACPA positivity, DAS28 and CRP were further included in the model, serum Helix III was still associated with progression with slightly lower OR, which remained significant for progression of erosion and total damage. Next, patients were categorized in quintiles of baseline serum Helix III levels. There was a significant association between increased quintiles and total joint damage progression [OR (95% CI): 1.34 (1.16-1.56) per 1 quintile increase] after adjustment for gender, BMI and RA diagnosis. Patients with levels in the highest quintile had an odds-ratio (95% CI) of total joint damage progression at 5 years of 2.91 (1.79-4.73) compared to patients in the lowest 4 quintiles. When progression evaluation was based on erosion or JSN score, the highest quintile of Helix III was also significantly associated with increased progression (data not shown). After further adjustment for ACPA positivity, DAS28 and CRP, the risk of total damage progression slightly decreased but remained highly significant (OR: 2.41, p=0.003 for highest of baseline Helix III vs lowest 4 quintiles). FIG. 8 shows the 5-year likelihood of total joint damage progression associated with high serum levels of Helix III compared to that of the usual risk factors available in the study. The high-risk group was identified as patients with levels in the highest quintile for all quantitative variables, i.e., Helix III, DAS28, and CRP. The largest odds-ratio of progression was observed for high Helix III, followed by ACPA positivity and then increased CRP and DAS28. When baseline Helix III levels were classified in tertiles or quartiles, there was also a significant relationship between increased levels and progression (data not shown). When the analysis was restricted to patients with a diagnosis of RA, similar results were obtained with an odds-ratio (95% CI) of 5-year progression of total damage of 1.47 (1.17-1.85) for each SD increase of baseline Helix III, after adjustment for gender and BMI. The odds-ratio approached significance (p=0.057) after further adjustment for ACPA positivity, DAS28 and CRP (data not shown).

### 1-Year Progression

As shown on Table 9, when analyzed as a continuous variable, baseline serum Helix III was significantly associated with the risk of erosion, JSN and total damage progression (OR: 1.36 to 1.89; p<0.0001 for each SD increase of Helix III after adjustment for gender, BMI and RA diagnosis). The association was highest for bone erosion progression. After further adjustment for ACPA positivity, DAS28 and CRP, the association remained highly significant for bone erosion and total damage progression, but not for JSN progression (Table 9).

### Combination of Risk Factors

Among the known risk factors of progression in RA, bone erosion is one of the strongest and in this study was only slightly associated with serum Helix III at baseline suggesting almost complete independence of these two risk factors. When serum Helix III (either as a continuous variable or in quintiles) and bone erosion were included in the same logistic regression model, both serum Helix III (p<0.0001) and erosion (p<0.001) were significantly associated with 5-year total damage progression, after adjustment for gender, BMI and RA diagnosis. The likelihood of progression in patients with both levels of Helix III in the highest quintile and presenting with bone erosion was more than 2-fold higher than the odds-ratio observed in patients with either high Helix III alone or bone erosion alone (FIG. 8). Association of high serum Helix III with the other risk factors, i.e., ACPA positivity, DAS28 or CRP did not improve the risk prediction of each of the variables alone.

### Sensitivity, Specificity and Likelihood of Positive Results of Baseline Helix III and Others Risk Factors to Predict 5-Year Radiographic Progression.

As shown on Table 10, the highest specificity and sensitivity figures were observed for serum Helix III. For each parameter, at the cut-off value used for defining high risk, the specificity was high (from 84 to 86%), but the sensitivity was modest (27 to 32%), except for ACPA for which both the specificity and sensitivity were moderate (63 and 60%, respectively). We also calculated the LR which is the most adequate index to directly compare the predictive value of different parameters at the level of the individual patient. The LR+ was highest for Helix III (2.3), followed by erosion (2.0). For the other risk factors (CRP, DAS and ACPA), the LR+ were all lower than 2.0 (from 1.5 to 1.8). When Helix III was combined with erosion, the LR+ increased substantially to reach 7.2 (FIG. 9). The combination of Helix III with ACPA was associated with a modest increase in the LR+ (3.4)

### Conclusion

Increased serum Helix III is associated with a higher risk of structural progression, independently of major risk factors. Helix III may be useful in association with bone erosion to identify patients with early arthritis at higher risk.

TABLE 7
Baseline characteristics of the 788 patients with early arthritis

| Variables | Mean (SD) | | Median (5-95 pct) | |
|---|---|---|---|---|
| RA positive patients | 643 | (82%) | | |
| (n, %) | 40 | (13) | 51 | (25-79) |
| Age (yr.) | | | | |
| Women (n, %) | 601 | (76%) | | |
| BMI (kg/cm²) | 25.0 | (4.6) | 24.3 | (19.0-42.9) |
| Symptom duration | 7.1 | (8.5) | 4.9 | (1.6-24) |
| (month) | | | | |
| DAS28-ESR | 5.11 | (1.32) | 5.09 | (1.58-8.66) |
| C-reactive protein | 22.3 | (34) | 9 | (1-92) |
| (mg/ml) | 308 | (39%) | | |
| ACPA positive patients | | | | |
| (n, %) | | | | |
| Erosion mSS | 0.61 | (2.17) | 0 | (0-25) |
| Narrowing mSS | 2.62 | (4.00) | 1 | (0-11) |
| Total mSS | 3.21 | (5.08) | 1.5 | (0-12) |
| Serum Helix III (ng/ml) | 201 | (144) | 189 | (24-472) |

RA: rheumatoid arthritis,
BMI: body mass index,
DAS28-ESR: disease activity score 28 using erythrocyte sedimentation rate (ESR),
ACPA: anti-citrullinated protein antibodies,
mSS: van der Heidje modified Sharp score

TABLE 8
Baseline serum Helix III and the 5-year risk of progression in patients
with early arthritis

| | Odds-ratio (95% CI) of progression for each | | | |
|---|---|---|---|---|
| | SD increase of baseline Helix III | | | |
| 5-year | Adj. for gender, | | Additionally, adj. for | |
| progression in | BMI, RA diagnosis | | ACPA, DAS28, CRP | |
| Erosion | 1.55 | (1.12-2.14) | 1.51 | (1.00- 2.30) |
| Joint space | 1.54 | (1.14-1.74) | 1.27 | (0.97-1.65) |
| narrowing | | | | |
| Total damage | 1.51 | (1.21-1.87) | 1.34 | (1.01-1.76) |

TABLE 9
Baseline serum Helix III and the 1-year risk of progression in
patients with early arthritis

| | Odds-ratio (95% CI) of progression for each | |
|---|---|---|
| | SD increase of baseline Helix III | |
| 1-year | Adj. for gender, | Additionally, adj. for |
| progression in | BMI, RA diagnosis | ACPA, DAS28, CRP |
| Erosion | 1.89 (1.45-2.47) | 1.60 (1.16-2.20) |
| Joint space | 1.36 (1.15-1.62) | 1.18 (0.96-1.46) |
| narrowing | | |
| Total damage | 1.47 (1.23-1.76) | 1.28 (1.03-1.60) |

TABLE 10
Accuracy of baseline serum Helix III, CRP, DAS 28 and ACPA for
predicting 5 year-radiographic progression in patients with early arthritis.
The table shows for each baseline prognostic factor, the specificity, the
sensitivity and the likelihood ratio for positive test (LR+)

| | Specificity | Sensitivity | |
|---|---|---|---|
| Baseline predictor | (%) | (%) | LR+ |
| Helix III, highest | 86 | 32 | 2.3 |
| quintile | | | |
| Erosion, ≥1 mSS* | 86 | 29 | 2.0 |
| CRP, highest | 84 | 24 | 1.5 |
| quintile | | | |
| DAS 28, highest | 84 | 28 | 1.8 |
| quintile | | | |
| ACPA, >50 unit/L | 63 | 60 | 1.6 |

*mSS: van der Heijde modified Sharp score

### REFERENCES

1. Smolen J S, Aletaha D, McInnes I B. Rheumatoid arthritis. Lancet. 2016; 388(10055):2023-38.
2. Hitchon C A, El-Gabalawy H S. The synovium in rheumatoid arthritis. Open Rheumatol J. 2011; 5:107-14.
3. Aletaha D, Smolen J S. Diagnosis and Management of Rheumatoid Arthritis: A Review. JAMA. 2018; 320(13):1360-72.
5. Garnero P, Landewe R, Chapurlat R D. The role of biochemical markers of joint tissue remodelling to predict progression and treatment efficacy in inflammatory rheumatic diseases. Rheumatology (Oxford). 2020; 59(6):1207-17.
6. Fex E, Jonsson K, Johnson U, Eberhardt K. Development of radiographic damage during the first 5-6 yr of rheumatoid arthritis. A prospective follow-up study of a Swedish cohort. Br J Rheumatol. 1996; 35(11):1106-15.
7. Christgau S, Garnero P, Fledelius C, Moniz C, Ensig M, Gineyts E, et al. Collagen type II C-telopeptide fragments as an index of cartilage degradation. Bone. 2001; 29(3):209-15.
8. Charni N, Juillet F, Garnero P. Urinary type II collagen helical peptide (HELIX-II) as a new biochemical marker of cartilage degradation in patients with osteoarthritis and rheumatoid arthritis. Arthritis Rheum. 2005; 52(4):1081-90.
9. Garnero P, Landewe R, Boers M, Verhoeven A, Van Der Linden S, Christgau S, et al. Association of baseline levels of markers of bone and cartilage degradation with long-term progression of joint damage in patients with early rheumatoid arthritis: the COBRA study. Arthritis Rheum. 2002; 46(11):2847-56.
10. Landewe R, Geusens P, Boers M, van der Heijde D, Lems W, te Koppele J, et al. Markers for type II collagen breakdown predict the effect of disease-modifying treatment on long-term radiographic progression in patients with rheumatoid arthritis. Arthritis Rheum. 2004; 50(5):1390-9.
11. Kiener H P, Karonitsch T. The synovium as a privileged site in rheumatoid arthritis: cadherin-11 as a dominant player in synovial pathology. Best Pract Res Clin Rheumatol. 2011; 25(6):767-77.
12. Schneider M, Voss B, Rauterberg J, Menke M, Pauly T, Miehlke R K, et al. Basement membrane proteins in synovial membrane: distribution in rheumatoid arthritis and synthesis by fibroblast-like cells. Clin Rheumatol. 1994; 13(1):90-7.
13. Lovell C R, Nicholls A C, Jayson M I, Bailey A J. Changes in the collagen of synovial membrane in rheumatoid arthritis and effect of D-penicillamine. Clin Sci Mol Med. 1978; 55(1):31-40.
14. Revell P A, al-Saffar N, Fish S, Osei D. Extracellular matrix of the synovial intimal cell layer. Ann Rheum Dis. 1995; 54(5):404-7.
15. Weiss J B, Shuttleworth C A, Brown R, Sedowfia K, Baildam A, Hunter J A. Occurrence of type III collagen in inflamed synovial membranes: a comparison between non rheumatoid, rheumatoid, and normal synovial collagens. Biochem Biophys Res Commun. 1975; 65(3):907-12.
16. Adam M, Vitasek R, Deyl Z, Felsch G, Musilova J, Olsovska Z. Collagen in rheumatoid arthritis. Clin Chim Acta. 1976; 70(1):61-9.
17. Garnero P, Gineyts E, Christgau S, Finck B, Delmas P D. Association of baseline levels of urinary glucosyl-galactosyl-pyridinoline and type II collagen C-telopeptide with progression of joint destruction in patients with early rheumatoid arthritis. Arthritis Rheum. 2002; 46(1):21-30.
18. Gineyts E, Garnero P, Delmas P D. Urinary excretion of glucosyl-galactosyl pyridinoline: a specific biochemical marker of synovium degradation. Rheumatology (Oxford). 2001; 40(3):315-23.
16. Gineyts E, Millet M, Borel O et al. Serum SynCol as a new biochemical marker of synovial tissue turnover in patients with rheumatoid arthritis. Art Res Ther (in press)
17. Combe B, Benessiano J, Berenbaum F et al. The ESPOIR cohort: a ten-year follow-up of early arthritis in France: methodology and baseline characteristics of the 813 included patients. Joint Bone Spine 2007; 74:440-5.
18. Combe B, Landewe R, Lukas C et al. EULAR recommendations for the management of early arthritis: report of a task force of the European Standing Committee for International Clinical Studies Including Therapeutics (ESCISIT). Ann Rheum Dis 2007; 66:34-45.
19. Prevoo M L, van 't Hof M A, Kuper H H, et al. Modified disease activity scores that include twenty-eight-joint counts. Development and validation in a prospective longitudinal study of patients with rheumatoid arthritis. Arthritis Rheum 1995; 38:44-48.
20. Aletaha D, Neogi T, Silman A J et al: Rheumatoid arthritis classification criteria: an American College of Rheumatology/European League Against Rheumatism collaborative initiative. Arthritis Rheum 2010, 2010:2569-2581.
21. van der Heijde D M, van Riel P L, Nuver-Zwart I H, et al. Effects of hydroxychloroquine and sulphasalazine on progression of joint damage in rheumatoid arthritis. Lancet 1989; 1:1036-1038.
22. Mouterde G, Lukas C, Logeart I et al. Predictors of radiographic progression in the ESPOIR cohort: the season of first symptoms may influence the short-term outcome in early arthritis. Ann Rheum Dis. 2011; 70:1251-6

1-12. (canceled)

13. An in vitro method for diagnosing a collagen degradation associated disease in a subject in need thereof and treating the subject comprising the following steps:
i)
i) detecting and/or quantifying the level of Helix-III peptide in a biological sample from the subject,
ii) determining that the level is higher than a reference value and
iii) administering to the subject one or more of a nonsteroidal anti-inflammatory drug (NSAID), a corticosteroid, a disease-modifying antirheumatic drug (DMARD), a biologic response modifier, a targeted synthetic DMARD and/or a drug selected from the group consisting of amiodarone, chlorpromazine, tolbutamide, isoniazid, methyldopa and oxyphenisatine.

14. The method according to claim 13 wherein the collagen degradation associated disease is selected from the group consisting of: osteoporosis, Paget's disease, a bone tumours, drug-related bone loss, osteoarthritis, rheumatoid arthritis, juvenile arthritis, psoriatic arthritis, spondyloarthropathies, and fibrosis.

15. An in vitro method for monitoring collagen degradation associated disease progression in a subject in need thereof and treating the subject comprising the steps of:
i)
detecting and/or quantifying the level of Helix-III peptide in said a biological sample from the subject,
ii) determining that the level is higher than a reference value and
iii) administering to the subject one or more of a nonsteroidal anti-inflammatory drug (NSAID), a corticosteroid, a disease-modifying antirheumatic drug (DMARD), a biologic response modifier, a targeted synthetic DMARD and/or a drug selected from the group consisting of amiodarone, chlorpromazine, tolbutamide, isoniazid, methyldopa and oxyphenisatine.

16. The method according to claim 13, further comprising determining, the level of bone erosion in the subject.

17. An in vitro method for predicting whether a subject will achieve a response to a collagen degradation associated disease treatment and treating the subject comprising the steps of:
i)
i) detecting and/or quantifying the level of Helix-III peptide in a biological sample obtained from the subject, and
ii) determining that the level is lower than a reference value and
iii) administering to the subject one or more of a nonsteroidal anti-inflammatory drug (NSAID), a corticosteroid, a disease-modifying antirheumatic drug (DMARD), a biologic response modifier, a targeted synthetic DMARD and/or a drug selected from the group consisting of amiodarone, chlorpromazine, tolbutamide, isoniazid, methyldopa and oxyphenisatine.

18. The method according to claim 13, wherein the biological sample is a serum sample, a plasma sample, a urine sample or a synovial fluid sample.

19. The method according to claim 13, wherein the method is performed by an enzyme-labeled and mediated immunoassays.

20. The method according to claim 13, wherein the method is performed by a competitive polyclonal antibody-based enzyme-linked immunosorbent assay (ELISA).

21. The method according to claim 13, wherein,
the NSAID is ibuprofen or naproxen sodium;
the corticosteroid is prednisone or prednisolone;
the DMARD is methotrexate, leflunomide, hydroxychloroquine or sulfasalazine; the biologic response modifier is abatacept, adalimumab, anakinra, certolizumab, etanercept, golimumab, infliximab, rituximab, sarilumab or tocilizumab; and
the targeted synthetic DMARD is baricitinib, tofacitinib, and upadacitinib or filgotinib.

22. A kit for performing the method of claim 13, said kit comprising:
a solid support,
a binding partner against an epitope comprising SEQ ID NO: 1, and
instructions for use.

23. A computer-implemented method for diagnosing collagen degradation associated disease in a subject according to claim 13, comprising the following steps:
i) contacting a biological sample with a binding partner against an epitope comprising SEQ ID NO: 1;
ii) quantifying the level of Helix-III peptide in said biological sample;
iii) incorporating said quantified value in a software with clinical parameters; and
iv) concluding that the subject suffers from a collagen degradation associated disease when the level of Helix-III peptide associated with clinical parameters determined at step iii) is higher than the reference value or concluding that the subject does not suffer from a collagen degradation associated disease when the level of Helix-III peptide associated with clinical parameters determined at step iii) is lower than the reference value.

24. The method of claim 13, wherein the step of detecting and/or quantifying is performed by contacting the biological sample with an antibody that recognizes and binds to:
an epitope comprising at least 7 amino acid residues from SEQ ID NO:1, or from a sequence sharing at least 90% of identity to SEQ ID NO: 1; or
an epitope comprising the amino acid sequence as set forth in SEQ ID NO: 1 or an amino acid sequence sharing at least 90% of identity to SEQ ID NO: 1, or an epitope consisting of SEQ ID NO:1.

25. The method according to claim 24, wherein the epitope comprising at least 7 amino acid residues from SEQ ID NO:1 recognizes and binds to a sequence selected from the group consisting of SEQ ID NO:2, SEQ ID NO: 3, SEQ ID NO: 4, SEQ ID NO: 5, SEQ ID NO: 6, SEQ ID NO:7 and SEQ ID NO: 8.

26. The method according to claim 24, which is conjugated with a detectable label.

27. The method according to claim 26, wherein the detectable label is a radioisotope, a fluorescent label, a chemiluminescent label, an enzyme label, or a bioluminescent label.

28. The method according to claim 27, wherein the detectable label is β-galactosidase, glucose oxidase, peroxidase or alkaline phosphatase.


SEQUENCE LISTING (8 sequences)

SEQ ID NO: 1 - protein, 7 aa, synthetic construct
PGPPGTS

SEQ ID NO: 2 - protein, 11 aa, synthetic construct
  MOD_RES 8: note = HYDROXYPROLINE
PPGPPGPPGTS

SEQ ID NO: 3 - protein, 11 aa, synthetic construct
PPGPPGPPGTS

SEQ ID NO: 4 - protein, 10 aa, synthetic construct
  MOD_RES 8: note = HYDROXYPROLINE
PPGPPGPPGT

SEQ ID NO: 5 - protein, 12 aa, synthetic construct
  MOD_RES 8: note = HYDROXYPROLINE
PPGPPGPPGTSG

SEQ ID NO: 6 - protein, 11 aa, synthetic construct
PSGPPGKDGTS

SEQ ID NO: 7 - protein, 11 aa, synthetic construct
LQGPPGPPGTS

SEQ ID NO: 8 - protein, 11 aa, synthetic construct
SDGAPGPPGTS